(19) United States
(12) Patent Application Publication
(10) Pub. No.: US 2014/0024981 A1
(43) Pub. Date: Jan. 23, 2014

(54) WEARABLE VIBRATORY STIMULATION DEVICE AND OPERATIONAL PROTOCOL THEREOF

(71) Applicants: Chang Mook CHUN, Bucheon-si (KR); Choong Hyun KIM, Seoul (KR); Jun Ho CHOI, Seoul (KR); Seung Jong KIM, Seoul (KR)

(72) Inventors: Chang Mook CHUN, Bucheon-si (KR); Choong Hyun KIM, Seoul (KR); Jun Ho CHOI, Seoul (KR); Seung Jong KIM, Seoul (KR)

(73) Assignee: KOREA INSTITUTE OF SCIENCE AND TECHNOLOGY, Seoul (KR)

(21) Appl. No.: 13/888,678

(22) Filed: May 7, 2013

(30) Foreign Application Priority Data
Jul. 23, 2012 (KR) 10-2012-0080024

(51) Int. Cl. A61H 1/00; A61B 5/11

(52) U.S. Cl. CPC A61H 1/00; A61B 5/112
USPC 601/69; 601/46

(57) ABSTRACT

The wearable vibratory stimulation device includes: a measuring instrument for obtaining data relating to a body motion of a user who wears the vibratory stimulation device; a walking pattern database for storing normal walking pattern data collected by measuring general persons having normal walking patterns and information about an inherent walking pattern analysis result of a specific user; a controller for analyzing the body motion information of the user, transmitted from the measuring instrument, by comparing with the walking pattern data stored in the walking pattern database, and generating a stimulation signal to be applied to a specific body portion of the user; and an exciting unit for applying a vibratory stimulation to the specific body portion in response to the stimulation signal transmitted from the controller.

### CROSS-REFERENCE TO RELATED APPLICATION

This application claims priority to Korean Patent Application No. 10-2012-0080024, filed on Jul. 23, 2012, and all the benefits accruing therefrom under 35 U.S.C. §119, the contents of which in its entirety are herein incorporated by reference.

### BACKGROUND

1. Field

The present disclosure relates to a wearable vibratory stimulation device and an operational protocol thereof, and more particularly, to a wearable vibratory stimulation device and an operational protocol thereof, which helps walking of a person who experiences inconvenience in walking.

2. Description of the Related Art

In order to help walking of persons who experience inconvenience in walking as a result of graying or nervous diseases or due to paralysis of a half body or muscles of a part of the body, various stimulation methods are being developed. The convention stimulation methods give fatigue to the muscle of a user, which demands gradually stronger stimulation or causes the user not to respond to the stimulation.

As conventional techniques for applying vibratory stimulation to a human body, Korean Unexamined Patent Publication No. 10-2007-0073566 discloses a device for measuring a body composition of a user and providing vibratory stimulation suitable for a body condition corresponding to body composition to the user. In addition, Korean Unexamined Patent Publication No. 10-2003-0069944 discloses an athletic machine using vibration and electric stimulation, which vibrates a lower body through the feet, vibrates an upper body through the handles, massages the waste by vibrating a belt, generates predetermined vibration, ultrasonic wave and low-frequency wave through probes provided at belts worn on the abdomen, the forearms, and the thighs, and allows the user to inhale anions.

In addition, Korean Unexamined Patent Publication No. 10-2004-0094510 provides a portable electromyogram-adjusting electric stimulator, which maximizes the treatment efficiency by allowing a patient to use the function of the electric stimulator together with his power without being limited to an existing treatment method depending on only the force of an electric stimulator by applying an electric stimulation to contract the muscle when a patent applies a force over a criterion electromyogram level. In addition, Korean Unexamined Patent Publication No. 10-2009-0089528 discloses a robot helper which allows a patient to evaluate his/her health by himself and recover from diseases by using a motor, an electrode and an electromyogram-functional electric stimulator to help muscular strength reinforcement and nervous recovery and by helping the patient to make bio feedback through the electromyogram-functional electric stimulator.

However, the conventional techniques described above give stimulation to a person by means of vibration in order and thus apply vibratory stimulation to a human body to enhance athletic effects or massage the user, but fails to suggest a method for correcting walking by applying vibratory stimulation to a specific portion of a human body in order to help walking of a user who experiences inconvenience in walking.

### RELATED LITERATURES

Patent Literature

Korean Unexamined Patent Publication No. 10-2007-0073566, (DMBH Ltd.) Jul. 10, 2007

Korean Unexamined Patent Publication No. 10-2003-0069944 (CHO, Seung-Hyeon) Aug. 27, 2003

Korean Unexamined Patent Publication No. 10-2004-0094510 (Cybermedic Ltd., PARK, Byeong-Rim) Nov. 10, 2004

Korean Unexamined Patent Publication No. 10-2009-0089528 (Industry Academy Cooperation Foundation of Yonsei University) Aug. 24, 2009

### SUMMARY

The present disclosure is directed to providing a wearable vibratory stimulation device and an operational protocol thereof, which may be easily worn by patients who experience inconvenience in walking due to muscle rigidity or stiffness caused by brain-nervous system diseases, particularly patients who feel difficulty in walking due to the foot drop, to help walking.

In one aspect, there is provided a wearable vibratory stimulation device, which includes: a measuring instrument for obtaining data relating to a body motion of a user who wears the vibratory stimulation device; a walking pattern database for storing normal walking pattern data collected by measuring general persons having normal walking patterns and information about an inherent walking pattern analysis result of a specific user; a controller for analyzing the body motion information of the user, transmitted from the measuring instrument, by comparing with the walking pattern data stored in the walking pattern database, and generating a stimulation signal to be applied to a specific body portion of the user; and an exciting unit for applying a vibratory stimulation to the specific body portion in response to the stimulation signal transmitted from the controller.

The wearable vibratory stimulation device may further include a motion capturing unit for capturing a motion of the user by using sensors attached to a plurality of body portions of the user.

In case of determining the walking pattern of the user is an abnormal motion, the controller may determine the kind of a stimulation signal, a stimulation pattern and a stimulation time point required for improving the motion of the corresponding body portion, and generate a stimulation signal.

The exciting unit may apply the vibratory stimulation to the muscle or tendon of the body by using an eccentric mass motor or a piezo-actuator.

The wearable vibratory stimulation device may further include a low-pass filter for removing noise from the signal relating to the user body motion information, transmitted from the measuring instrument to the controller.

The controller may include a Kalman filter or a particle filter in order to estimate a walking pattern of the user from the signal relating to the user body motion information having noise.

In another aspect, there is provided an operational protocol of a wearable vibratory stimulation device, which includes: measuring, by a measuring instrument, a motion of a user who wears the vibratory stimulation device and obtaining data relating to a body motion of the user; transmitting the data obtained by the measuring instrument to a controller; analyzing, by the controller, the data relating to the body motion of the user, transmitted from the measuring instrument, by comparing with a walking pattern data stored in a walking pattern database, to determine whether the walking pattern of the user is normal; in the case the controller determines that the walking pattern of the user is abnormal, determining the kind of a stimulation signal, a stimulation pattern and a stimulation time point and generating a stimulation signal in order to correct the walking pattern; and applying, by an exciting unit, a vibratory stimulation to a specific body portion of the user in response to the stimulation signal transmitted from the controller.

The controller may receive the data relating to the body motion of the user by using a motion capturing unit which captures a motion of the user by using sensors attached to a plurality of body portions of the user.

The exciting unit may apply the vibratory stimulation to the muscle or tendon of the body by using an eccentric mass motor or a piezo-actuator.

The wearable vibratory stimulation device and the operational protocol thereof according to the present disclosure may correct a walking pattern of a user by analyzing body motion information of the user and applying effective vibratory stimulation to a local muscle at an accurate time point, for the patients feeling difficulty in walking due to muscle rigidity or stiffness.

### BRIEF DESCRIPTION OF THE DRAWINGS

The above and other aspects, features and advantages of the disclosed exemplary embodiments will be more apparent from the following detailed description taken in conjunction with the accompanying drawings in which:

FIG. 1 is a block diagram showing a wearable vibratory stimulation device according to an embodiment of the present disclosure; and

FIG. 2 is a flowchart for illustrating an operational protocol of the wearable vibratory stimulation device according to an embodiment of the present disclosure.

[Detailed Description of Main Elements]

100: wearable vibratory stimulation device

110: measuring instrument

120: walking pattern database

130: controller

140: exciting unit

150: motion capturing unit

### DETAILED DESCRIPTION

Hereinafter a wearable vibratory stimulation device and an operational protocol thereof according to an embodiment of the present disclosure will be described in detail with reference to the accompanying drawings.

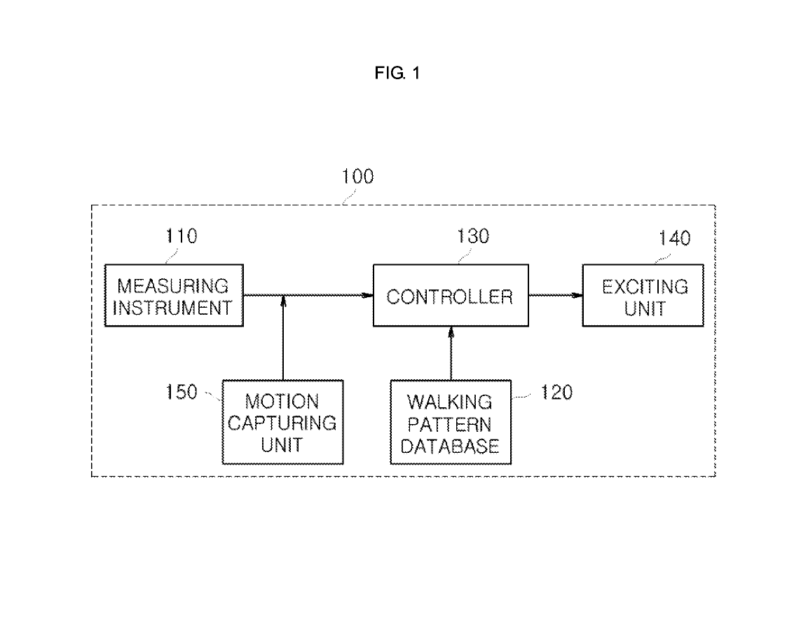

FIG. 1 is a block diagram showing a wearable vibratory stimulation device according to an embodiment of the present disclosure.

Referring to FIG. 1, a wearable vibratory stimulation device 100 of the present disclosure includes measuring instrument 110, a walking pattern database 120, a controller 130 and an exciting unit 140 and selectively further includes a motion capturing unit 150.

The measuring instrument 110 obtains data relating to a body motion of a user who wears the vibratory stimulation device 100, such as location, velocity, acceleration and direction. The measuring instrument 110 may be mounted to a specific body portion of the user, such as the spine, the waste, the knee joint, the ankle joint or the like, and may use a position sensor, a velocity sensor, an angular velocity sensor or the like in order to sense a walking pattern of the user. The data relating to the body motion of the user measured by the measuring instrument 110 is transmitted to the controller 130.

The walking pattern database 120 stores walking pattern data of normal persons and specific persons and provides the walking pattern data to the controller 130 so that the controller 130 may analyze the body motion information of the user, transmitted from the measuring instrument 110, based on the walking pattern data of normal persons and specific persons, and determine the kind of a stimulation signal, a stimulation pattern and a stimulation time point. The walking pattern database 120 may include both normal walking pattern data collected by measuring general persons having normal walking patterns and information about an inherent walking pattern analysis result of a specific person. In addition, the walking pattern database 120 may update information by receiving walking pattern data of normal persons and specific persons from an external device.

The controller 130 analyzes the user body motion information transmitted from the measuring instrument 110 or the motion capturing unit 150 by comparing with the walking pattern data stored in the walking pattern database 120 and generates a stimulation signal to be applied to a specific body portion of the user.

The controller 130 may be configured as a small computer or a mobile device. And, the controller 130 analyzes a motion of each portion of the body by using the user body motion information transmitted from the measuring instrument 110 or the motion capturing unit 150, then comparing it with the walking pattern data, and determines whether the walking of the user is normal. In case of determining that the walking pattern of the user is an abnormal motion, the controller 130 determines the kind of a stimulation signal, a stimulation pattern and a stimulation time point, required for improving the motion of the corresponding body portion, and generates a stimulation signal. The stimulation signal generated by the controller 130 is transmitted to the exciting unit 140 through wired/wireless equipment to apply a stimulation to a predetermined tendon portion.

The wearable vibratory stimulation device 100 of the present disclosure may further include a low-pass filter (not shown) in order to remove noise from the signal relating to the user body motion information, transmitted from the measuring instrument 110 to the controller 130, when the walking is analyzed. In addition, the controller 130 may include a Kalman filter (not shown), a particle filter (not shown) or the like in order to estimate the walking pattern of the user more accurately from the signal relating to the user body motion information.

The wearable vibratory stimulation device 100 according to another embodiment of the present disclosure may further include a motion capturing unit 150 for capturing a motion of the user by using sensors attached to a plurality of body portions of the user. The wearable vibratory stimulation device 100 of the present disclosure may utilize the motion capturing unit 150 at a hospital or rehabilitation center and operate by replacing the signal of the measuring instrument 110 or transmitting the body motion information of the user measured by the motion capturing unit 150 to the controller 130 together with the measuring instrument 110.

The exciting unit 140 applies a vibratory stimulation to a specific body portion of the user, for example, to a specific muscle or tendon portion in response to the stimulation signal received from the controller 130. The exciting unit 140 may apply the vibratory stimulation to a muscle or tendon portion of the body by using an eccentric mass motor or a piezo-actuator. According to research results on the brain-nervous system, if a minute vibratory stimulation of several ten Hz is applied to a tendon portion of the muscle, a sensory nervous system sensing the degree of contraction of muscle receives the stimulation, and the influence is transferred to the cerebrum to lessen the paralysis of the muscle, thereby allowing a person to actively move in a more stable way. Therefore, if a vibratory stimulation is applied to a specific body portion of the user by using the exciting unit 140 in order to maintain a walking pattern of the user in a normal state, the walking of the user may be corrected more smoothly and more stably.

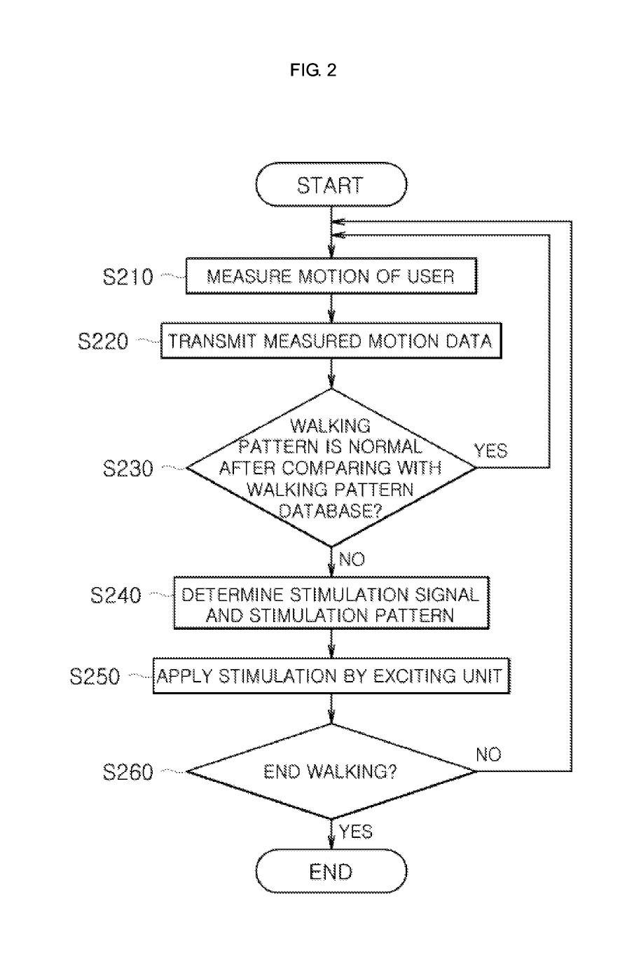

FIG. 2 is a flowchart for illustrating an operational protocol of the wearable vibratory stimulation device according to an embodiment of the present disclosure.

Referring to FIG. 2, first, the measuring instrument 110 or the motion capturing unit 150 measures a motion of a user who is wearing the vibratory stimulation device 100, and obtains data relating to a body motion, such as user location, velocity, acceleration, direction or the like (S210), and the measured motion data is transmitted to the controller 130 (S220).

Subsequently, the controller 130 analyzes the measured motion data, transmitted from the measuring instrument 110 or the motion capturing unit 150, to by comparing with the walking pattern data stored in the walking pattern database 120, and determines whether the walking pattern of the user is normal (S230). If the walking pattern of the user is normal, the measuring instrument 110 or the motion capturing unit 150 keeps measuring a motion of the user, while, if the walking pattern of the user is abnormal, in order to correct the walking pattern, the kind of a stimulation signal as well as a stimulation pattern and a stimulation time point are determined to generate a stimulation signal (S240).

Successively, the stimulation signal generated by the controller 130 is transmitted to the exciting unit 140 through wired/wireless equipment, and the exciting unit 140 applies a vibratory stimulation to a specific body portion of the user in response to the stimulation signal transmitted from the controller 130 (S250).

Subsequently, it is determined whether the user ends walking (S260), and if the walk keeps walking, the processes are repeated in order from S210.

While the exemplary embodiments have been shown and described, it will be understood by those skilled in the art that various changes in form and details may be made thereto without departing from the spirit and scope of the present disclosure as defined by the appended claims.

What is claimed is:

1. A wearable vibratory stimulation device, comprising:
a measuring instrument for obtaining data relating to a body motion of a user who wears the vibratory stimulation device;
a walking pattern database for storing normal walking pattern data collected by measuring general persons having normal walking patterns and information about an inherent walking pattern analysis result of a specific user;
a controller for analyzing the body motion information of the user, transmitted from the measuring instrument, by comparing with the walking pattern data stored in the walking pattern database, and generating a stimulation signal to be applied to a specific body portion of the user; and
an exciting unit for applying a vibratory stimulation to the specific body portion in response to the stimulation signal transmitted from the controller.

2. The wearable vibratory stimulation device according to claim 1, further comprising a motion capturing unit for capturing a motion of the user by using sensors attached to a plurality of body portions of the user.

3. The wearable vibratory stimulation device according to claim 1, wherein, in case of determining the walking pattern of the user is an abnormal motion, the controller determines the kind of a stimulation signal, a stimulation pattern and a stimulation time point required for improving the motion of the corresponding body portion, and generates a stimulation signal.

4. The wearable vibratory stimulation device according to claim 1, wherein the exciting unit applies the vibratory stimulation to the muscle or tendon of the body by using an eccentric mass motor or a piezo-actuator.

5. The wearable vibratory stimulation device according to claim 1, further comprising a low-pass filter for removing noise from the signal relating to the user body motion information, transmitted from the measuring instrument to the controller.

6. The wearable vibratory stimulation device according to claim 1, wherein the controller includes a Kalman filter or a particle filter in order to estimate a walking pattern of the user from the signal relating to the user body motion information having noise.

7. An operational protocol of a wearable vibratory stimulation device, comprising:
measuring, by a measuring instrument, a motion of a user who wears the vibratory stimulation device and obtaining data relating to a body motion of the user;
transmitting the data obtained by the measuring instrument to a controller;
analyzing, by the controller, the data relating to the body motion of the user, transmitted from the measuring instrument, by comparing with a walking pattern data stored in a walking pattern database, to determine whether the walking pattern of the user is normal;
in the case the controller determines that the walking pattern of the user is abnormal, determining the kind of a stimulation signal, a stimulation pattern and a stimulation time point and generating a stimulation signal in order to correct the walking pattern; and
applying, by an exciting unit, a vibratory stimulation to a specific body portion of the user in response to the stimulation signal transmitted from the controller.

8. The operational protocol of a wearable vibratory stimulation device according to claim 7, wherein the controller is capable of receiving the data relating to the body motion of the user by using a motion capturing unit which captures a motion of the user by using sensors attached to a plurality of body portions of the user.

9. The operational protocol of a wearable vibratory stimulation device according to claim 7, wherein the exciting unit applies the vibratory stimulation to the muscle or tendon of the body by using an eccentric mass motor or a piezo-actuator.
